(19) United States
(12) Patent Application Publication
(10) Pub. No.: US 2008/0103575 A1
(43) Pub. Date: May 1, 2008

(54) Implantable medical elongated member including balloon fixation element

(75) Inventor: Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/591,447

(22) Filed: Oct. 31, 2006

(51) Int. Cl. A61N 1/00

(52) U.S. Cl. 607/117

(57) ABSTRACT

An implantable elongated member configured to deliver a therapy from a medical device to a target therapy delivery site includes an inflatable balloon fixation element composed at least in part of a biocompatible degradable material. A biocompatible fluid may be delivered to the balloon fixation element via an inflation lumen in order to inflate the balloon upon implantation of the elongated member to fix the elongated member proximate to the target therapy delivery site. The balloon fixation element may be formed of a degradable material and deteriorate over time, such as in response to fibrous tissue growth.

### TECHNICAL FIELD

The invention relates to medical device systems and, more particularly, to elongated members in medical device systems.

### BACKGROUND

Electrical stimulation systems may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. An electrical stimulation system typically includes one or more implantable medical leads coupled to a neurostimulator.

The implantable medical lead may be percutaneously or surgically implanted in a patient on a temporary or permanent basis such that at least one stimulation electrode is positioned proximate to a target stimulation site. The target stimulation site may be, for example, a nerve or other tissue site, such as a spinal cord, pelvic nerve, pudendal nerve, stomach, bladder, or within a brain or other organ of a patient, or within a muscle or muscle group of a patient. The one or more electrodes located proximate to the target stimulation site may deliver electrical stimulation therapy to the target stimulation site in the form electrical signals.

Electrical stimulation of a sacral nerve may eliminate or reduce some pelvic floor disorders by influencing the behavior of the relevant structures, such as the bladder, sphincter and pelvic floor muscles. Pelvic floor disorders include urinary incontinence, urinary urge/frequency, urinary retention, pelvic pain, bowel dysfunction, and male and female sexual dysfunction. The organs involved in bladder, bowel, and sexual function receive much of their control via the second, third, and fourth sacral nerves, commonly referred to as S2, S3 and S4 respectively. Thus, in order to deliver electrical stimulation to at least one of the S2, S3, or S4 sacral nerves, an implantable medical lead is implanted proximate to the sacral nerve(s).

Electrical stimulation of a peripheral nerve, such as stimulation of an occipital nerve, may be used to mask a patient's feeling of pain with a tingling sensation, referred to as paresthesia. Occipital nerves, such as a lesser occipital nerve, greater occipital nerve or third occipital nerve, exit the spinal cord at the cervical region, extend upward and toward the sides of the head, and pass through muscle and fascia to the scalp. Pain caused by an occipital nerve, e.g. occipital neuralgia, may be treated by implanting a lead proximate to the occipital nerve to deliver stimulation therapy.

In many electrical stimulation applications, it is desirable for a stimulation lead to resist migration following implantation. For example, it may be desirable for the electrodes disposed at a distal end of the implantable medical lead to remain proximate to a target stimulation site in order to provide adequate and reliable stimulation of the target stimulation site. In some applications, it may also be desirable for the electrodes to remain substantially fixed in order to maintain a minimum distance between the electrode and a nerve in order to help prevent inflammation to the nerve and in some cases, unintended nerve damage. Securing the implantable medical lead at the target stimulation site may minimize lead migration.

### SUMMARY

In general, the invention is directed toward an implantable medical elongated member that includes one or more inflatable balloon fixation elements to substantially fix the elongated member proximate to a target therapy site in a patient, as well as a method for implanting the implantable medical elongated member. The one or more balloon fixation elements are coupled to the elongated member. When the elongated member is implanted in the patient, the one or more balloon fixation elements are typically in a first, substantially deflated state. In order to inflate the balloon upon implantation in the patient, a fluid is delivered to the balloon via a fluid delivery lumen, thereby enabling the balloon to expand and engage with surrounding tissue at the target therapy delivery site. At least a portion of the balloon fixation element is composed of a biocompatible degradable material, such that once the elongated member is implanted in a patient, at least the portion of the balloon fixation element may degrade in vivo over time.

The elongated member is configured to be coupled to a medical device to deliver a therapy from the medical device to target therapy delivery site in a patient. The therapy may be electrical stimulation, drug delivery, or both. In one embodiment, the implantable medical elongated member is an implantable medical lead that is configured to be coupled to an external or implantable electrical stimulator. The electrical stimulator is configured to deliver electrical stimulation therapy to a target stimulation site in a patient via the lead, and more specifically, via at least one electrode disposed adjacent to a distal end of a lead body of the lead. In another embodiment, the elongated member is a catheter configured to deliver a fluid, such as pharmaceutical agents, insulin, pain relieving agents, gene therapy agents, or the like from an external or implantable fluid delivery device (e.g., a fluid reservoir and/or pump) to a target tissue site in a patient.

In another embodiment, the invention is directed toward an implantable elongated member comprising an elongated body configured to be coupled to a medical device to deliver a therapy from the medical device to a target therapy delivery site in a patient, an inflation lumen, and a balloon fixation element coupled to the elongated body and composed at least in part of a biocompatible degradable material. The balloon fixation element is configured to receive a fluid via the inflation lumen to expand from a first state to a second state.

In another embodiment, the invention is directed toward a system comprising a medical device and an elongated member. The elongated member comprises an implantable elongated body configured to be coupled to a medical device to deliver a therapy from the medical device to a target therapy delivery site in a patient, an inflation lumen, and a balloon fixation element coupled to the elongated body and composed at least in part of a biocompatible degradable material. The balloon fixation element is configured to receive a fluid via the inflation lumen to expand from a first state to a second state.

In yet another embodiment, the invention is directed toward a method comprising inserting an elongated member into the patient, wherein the elongated member includes a balloon fixation element mounted to the elongated member and composed at least in part of a biocompatible degradable material, advancing the elongated member to a target therapy delivery site to deploy the balloon fixation element into tissue of the patient proximate to the target therapy delivery site, and delivering a fluid to the balloon fixation element via an inflation lumen to inflate the balloon fixation element from a first state to a second state, wherein in the second state, the balloon fixation element engages with tissue at the target therapy delivery site.

A method comprising:

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A is a schematic perspective view of a therapy system, which includes an electrical stimulator coupled to an implantable medical lead, which has been implanted in a body of a patient proximate to a target stimulation site.

FIG. 1B illustrates the implantation of a stimulation lead at a location proximate to an occipital nerve.

FIG. 2 is a block diagram illustrating various components of an electrical stimulator and an implantable lead.

FIGS. 3A and 3B are perspective views of an exemplary neurostimulation lead that may be fixated to surrounding tissue to help prevent migration of the lead following implantation.

FIGS. 4A-4C are perspective views of leads including alternate configurations of an inflatable balloon fixation mechanism mounted on the body of a lead for fixing positions of leads in accordance with the invention.

FIGS. 5A and 5B are perspective views of an alternative inflation lumen configuration that may be used to inflate balloon fixation elements in accordance with one embodiment of the invention.

FIG. 6 is a flow diagram illustrating a process for percutaneously implanting a lead including a fixation mechanism in accordance with one embodiment of the invention.

### DETAILED DESCRIPTION

The present invention relates to an implantable medical elongated member including one or more balloon elements each including at least a portion that is formed of a biocompatible degradable material. The one or more balloon fixation elements are each configured to expand from a first, substantially deflated state to a second, substantially inflated state upon the introduction of a fluid into the one or more balloon fixation elements. For example, the one or more balloon elements may be expanded upon implantation of the elongated member in a patient, whereby upon expansion, the one or more balloon fixation members each extends from the elongated member and engages with surrounding tissue to substantially fix a position of the elongated member proximate to a target therapy delivery site. Because at least a portion of each of the one or more balloon fixation elements is formed at least in part of a degradable material, the balloon fixation element may be used to temporarily fix the elongated member until a sufficient amount of tissue encapsulates the elongated member to fix the elongated member.

Various embodiments of the elongated member may be applicable to different therapeutic applications. For example, the elongated member may be a stimulation lead, such as a neurostimulation lead, or a lead extension that is used to deliver electrical stimulation to a target stimulation site. In another embodiment, the elongated member may be a catheter that is placed to deliver a fluid, such as pharmaceutical agents, insulin, pain relieving agents, gene therapy agents, or the like from a fluid delivery device (e.g., a fluid reservoir or pump) to a target tissue site in a patient. The invention is applicable to any configuration or type of implantable elongated member that is used to deliver therapy to a site in a patient. For purposes of illustration, however, the disclosure will refer to a neurostimulation lead.

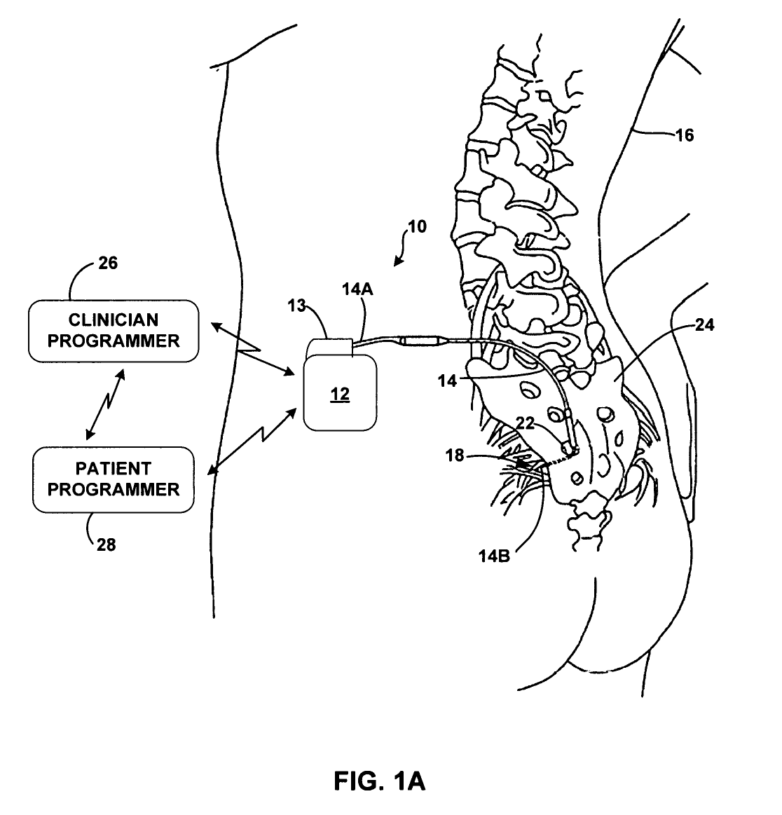

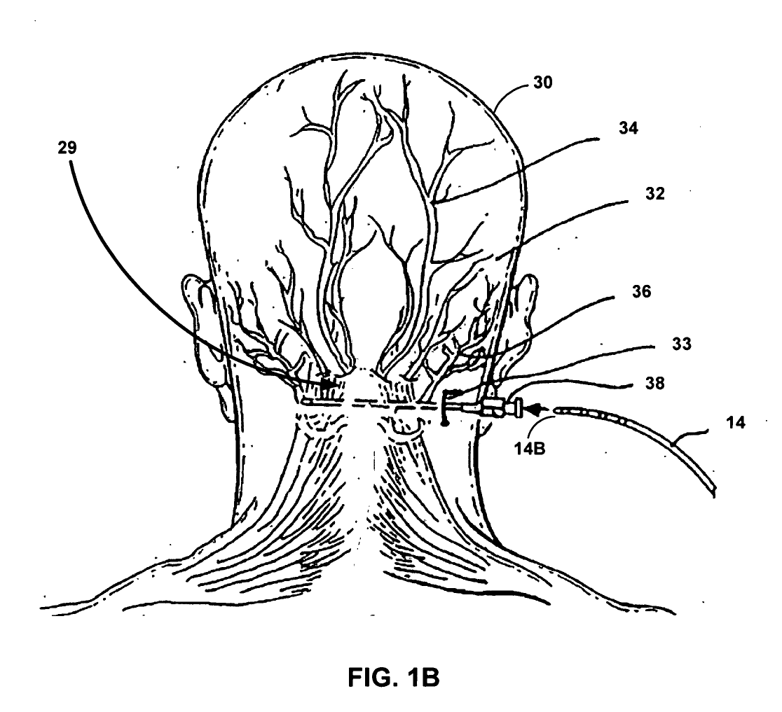

FIG. 1A is a schematic perspective view of therapy system 10, which includes electrical stimulator 12 coupled to neurostimulation lead 14. Electrical stimulator 12 provides a programmable stimulation signal (e.g., in the form of electrical pulses or substantially continuous-time signals) that is delivered to target stimulation site 18 by neurostimulation lead 14, and more particularly, via one or more stimulation electrodes carried by lead 14. In some embodiments, lead 14 may also carry one or more sense electrodes to permit electrical stimulator 12 to sense electrical signals from target stimulation site 18. Neurostimulator 12 may be either implantable or external. For example, electrical stimulator 12 may be subcutaneously implanted in the body of a patient 16 (e.g., in a chest cavity, lower back, lower abdomen, or buttocks of patient 16). In the example of FIG. 1A, electrical stimulator 12 is a neurostimulator that is implanted in patient 16 proximate to target stimulation site 18. Electrical stimulator 12 may also be referred to as a signal generator, and in the embodiment shown in FIG. 1A, electrical stimulator 12 may also be referred to as a neurostimulator. In some embodiments, neurostimulator 12 may be coupled to two or more leads, e.g., for bilateral or multi-lateral stimulation.

As described in greater detail below, lead 14 further includes a lead body and one or more expandable balloon fixation elements (not shown in FIG. 1A) coupled to the lead body. In a deflated state, each of the one or more balloon fixation elements may be substantially flush with an outer surface of the lead body of lead 14 so as not to protrude from the lead body or alternatively, each of the balloon fixation elements may protrude slightly from the lead body. Thus, when the one or more balloon fixation elements are in the deflated state, a profile of lead 14 is minimized. In an inflated state, each of the one or more balloon fixation elements extends from the lead body to engage with surrounding tissue to substantially fix a position of lead 14 proximate to target stimulation site 18. In the inflated state, each of the one or more balloon fixation elements protrudes further from the lead body than in the unexpanded state. Proximal end 14A of lead 14 may be both electrically and mechanically coupled to connector 13 of neurostimulator 12 either directly or indirectly (e.g., via a lead extension). In particular, conductors disposed in the lead body may electrically connect stimulation electrodes (and sense electrodes, if present) adjacent to distal end 14B of lead 14 to neurostimulator 12.

In the embodiment of therapy system 10 shown in FIG. 1A, target stimulation site 18 is proximate to the S3 sacral nerve, and lead 14 has been introduced into the S3 sacral foramen 22 of sacrum 24 to access the S3 sacral nerve. Stimulation of the S3 sacral nerve may help treat pelvic floor disorders, urinary control disorders, fecal control disorders, interstitial cystitis, sexual dysfunction, and pelvic pain. Therapy system 10, however, is useful in other neurostimulation applications. Thus, in alternate embodiments, target stimulation site 18 may be a location proximate to any of the other sacral nerves in body 16 or any other suitable nerve in body 16, which may be selected based on, for example, a therapy program selected for a particular patient. For example, in other embodiments, therapy system 10 may be used to deliver neurostimulation therapy to a pudendal nerve, a perineal nerve, an occipital nerve (as shown in FIG. 1B) or other areas of the nervous system, in which cases, lead 14 would be implanted and substantially fixed proximate to the respective nerve.

Therapy system 10 also may include a clinician programmer 26 and a patient programmer 28. Clinician programmer 26 may be a handheld computing device that permits a clinician to program neurostimulation therapy for patient 16, e.g., using input keys and a display. For example, using clinician programmer 26, the clinician may specify neurostimulation parameters for use in delivery of neurostimulation therapy. Clinician programmer 26 supports telemetry (e.g., radio frequency telemetry) with neurostimulator 12 to download neurostimulation parameters and, optionally, upload operational or physiological data stored by neurostimulator 12. In this manner, the clinician may periodically interrogate neurostimulator 12 to evaluate efficacy and, if necessary, modify the stimulation parameters.

Like clinician programmer 26, patient programmer 28 may be a handheld computing device. Patient programmer 28 may also include a display and input keys to allow patient 16 to interact with patient programmer 28 and neurostimulator 12. In this manner, patient programmer 28 provides patient 16 with an interface for control of neurostimulation therapy by neurostimulator 12. For example, patient 16 may use patient programmer 28 to start, stop or adjust neurostimulation therapy. In particular, patient programmer 28 may permit patient 16 to adjust stimulation parameters such as duration, amplitude, pulse width and pulse rate, within an adjustment range specified by the clinician via clinician programmer 28, or select from a library of stored stimulation therapy programs.

Neurostimulator 12, clinician programmer 26, and patient programmer 28 may communicate via cables or a wireless communication, as shown in FIG. 1A. Clinician programmer 26 and patient programmer 28 may, for example, communicate via wireless communication with neurostimulator 12 using RF telemetry techniques known in the art. Clinician programmer 26 and patient programmer 28 also may communicate with each other using any of a variety of local wireless communication techniques, such as RF communication according to the 802.11 or Bluetooth specification sets, infrared communication, e.g., according to the IrDA standard, or other standard or proprietary telemetry protocols.

Therapy system 10 may also be used to provide stimulation therapy to other nerves of a patient. For example, as shown in FIG. 1B, lead 14 may be implanted and fixated with the one or more balloon fixation elements proximate to an occipital region 29 of patient 30 for stimulation of one or more occipital nerves. In particular, lead 14 may be implanted proximate to lesser occipital nerve 32, greater occipital nerve 34, and third occipital nerve 36.

In FIG. 1B, lead 14 is aligned to be introduced into introducer needle 38 and implanted and anchored or fixated with fixation elements proximate to occipital region 29 of patient 30 for stimulation of one or more occipital nerves 32, 34, and/or 36. A neurostimulator (e.g., neurostimulator 12 in FIG. 1A) may deliver stimulation therapy to any one or more of occipital nerve 32, greater occipital nerve 34 or third occipital nerve 36 via electrodes disposed adjacent to distal end 14B of lead 14. In alternate embodiments, lead 14 may be positioned proximate to one or more other peripheral nerves proximate to occipital nerves 32, 34, and 36 of patient 30, such as nerves branching from occipital nerves 32, 34, and 36, as well as stimulation of any other suitable nerves throughout patient 30, such as, but not limited to, nerves within a brain, stomach or spinal cord of patient 30.

Implantation of lead 14 may involve the subcutaneous placement of lead 14 transversely across one or more occipital nerves 32, 34, and/or 36 that are causing patient 30 to experience pain. In one example method of implanting lead 14 proximate to the occipital nerve, using local anesthesia, a vertical skin incision 33 approximately two centimeters in length is made in the neck of patient 30 lateral to the midline of the spine at the level of the C1 vertebra. The length of vertical skin incision 33 may vary depending on the particular patient. At this location, the skin and muscle of patient 30 are separated by a band of connective tissue referred to as fascia. Introducer needle 38 is introduced into the subcutaneous tissue, superficial to the fascia and muscle layer but below the skin. Occipital nerves 32, 34, and 36 are located within the cervical musculature and overlying fascia, and as a result, introducer needle 38 and, eventually, lead 14 are inserted superior to occipital nerves 32, 34, and 36.

Once introducer needle 38 is fully inserted, lead 14 may be advanced through introducer needle 38 and positioned to allow stimulation of the lesser occipital nerve 32, greater occipital nerve 34, third occipital nerve 36, and/or other peripheral nerves proximate to an occipital nerve. Upon placement of lead 14, introducer needle 38 may be removed.

Accurate lead placement may affect the success of occipital nerve stimulation. If lead 14 is located too deep, i.e., anterior, in the subcutaneous tissue, patient 30 may experience muscle contractions, grabbing sensations, or burning. Such problems may additionally occur if lead 14 migrates after implantation. Furthermore, due to the location of implanted lead 14 on the back of the neck of patient 30, lead 14 may be subjected to pulling and stretching that may increase the chances of lead migration. For these reasons, fixating lead 14 may be advantageous.

In alternate applications of lead 14, target stimulation site 18 may be a location proximate to any of the other sacral nerves in patient 16 or any other suitable nerve, organ, muscle, muscle group or another suitable tissue site in patient 16, which may be selected based on, for example, a therapy program selected for a particular patient. For example, therapy system 10 may be used to deliver neurostimulation therapy to a pudendal nerve, a perineal nerve or other areas of the nervous system, in which cases, lead 14 would be implanted and substantially fixed proximate to the respective nerve. As further examples, lead 14 may be positioned for temporary or chronic spinal cord stimulation for the treatment of pain, for peripheral neuropathy or post-operative pain mitigation, ilioinguinal nerve stimulation, intercostal nerve stimulation, gastric stimulation for the treatment of gastric mobility disorders and obesity, muscle stimulation (e.g., functional electrical stimulation (FES) of muscles), for mitigation of other peripheral and localized pain (e.g., leg pain or back pain), or for deep brain stimulation to treat movement disorders and other neurological disorders. Accordingly, although patient 16 and target stimulation site 18 of FIG. 1A are referenced throughout the remainder of the disclosure for purposes of illustration, a neurostimulation lead 14 in accordance with the invention may be adapted for use in a variety of electrical stimulation applications, including occipital nerve stimulation, as shown in FIG. 1B with respect to patient 30.

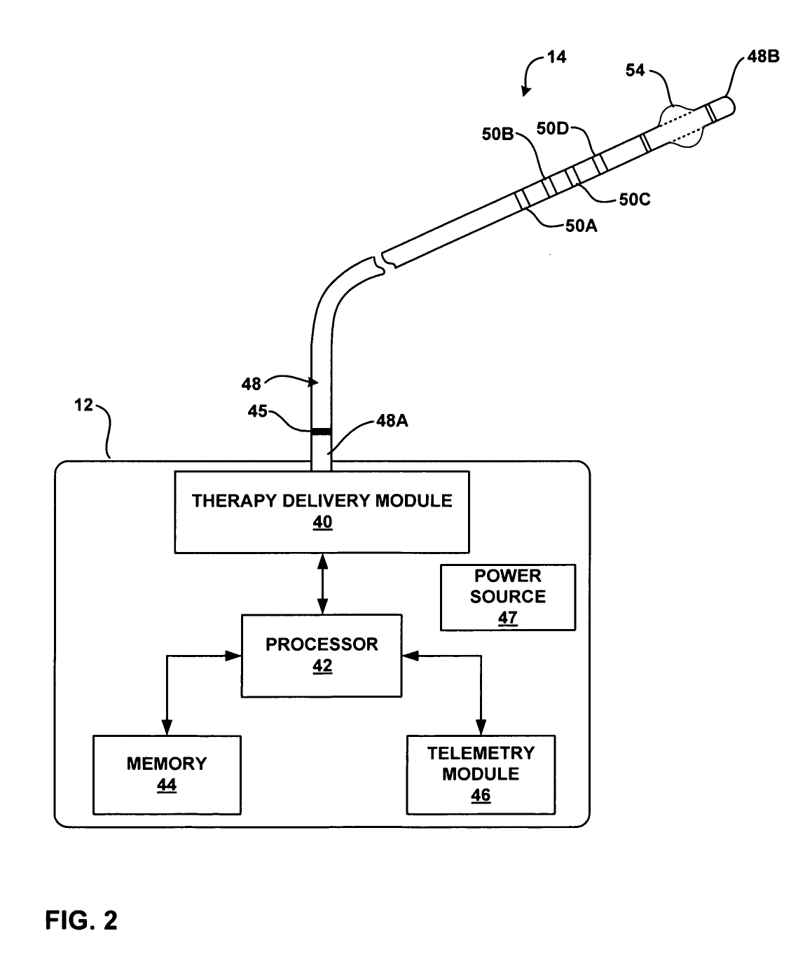

FIG. 2 is a block diagram illustrating various components of neurostimulator 12 and an implantable lead 14. Neurostimulator 12 includes therapy delivery module 40, processor 42, memory 44, telemetry module 46, and power source 47. In some embodiments, neurostimulator 12 may also include a sensing circuit (not shown in FIG. 2). Implantable lead 14 includes elongated lead body 48 extending between proximal end 48A and distal end 48B. Lead body 48 may be a cylindrical or may be a paddle-shaped (i.e., a “paddle” lead). Electrodes 50A, 50B, 50C, and 50D (collectively “electrodes 50”) are disposed on lead body 48 adjacent to distal end 48B of lead body 48.

In some embodiments, electrodes 50 may be ring electrodes. In other embodiments, electrodes 50 may be segmented or partial ring electrodes, each of which extends along an arc less than 360 degrees (e.g., 90-120 degrees) around the periphery of lead body 48. In embodiments in which lead 14 is a paddle lead, electrodes 50 may extend along one side of lead body 48. The configuration, type, and number of electrodes 50 illustrated in FIG. 2 are merely exemplary.

Electrodes 50 extending around a portion of the circumference of lead body 48 or along one side of a paddle lead may be useful for providing an electrical stimulation field in a particular direction/targeting a particular therapy deliver site. For example, in the electrical stimulation application shown in FIG. 1B, electrodes 50 may be disposed along lead body 48 such that the electrodes face toward occipital nerves 32, 34, and/or 36, or otherwise away from the scalp of patient 30. This may be an efficient use of stimulation because electrical stimulation of the scalp may not provide any therapy to patient 30. In addition, the use of segmented or partial ring electrodes 50 may also reduce the overall power delivered to electrodes 50 by neurostimulator 12 because of the efficient delivery of stimulation to occipital nerves 32, 34, and/or 36 (or other target stimulation site) by eliminating or minimizing the delivery of stimulation to unwanted or unnecessary regions within patient 30.

In embodiments in which electrodes 50 extend around a portion of the circumference of lead body 48 or along one side of a paddle lead, lead 14 may include one or more orientation markers 45 proximate to proximal end 14A that indicate the relative location of electrodes 50. Orientation marker 45 may be a printed marking on lead body 48, an indentation in lead body 48, a radiographic marker, or another type of marker that is visible or otherwise detectable (e.g., detectable by a radiographic device) by a clinician. Orientation marker 45 may help a clinician properly orient lead 14 such that electrodes 50 face the desired direction (e.g., toward occipital nerves 32, 34, and/or 36) within patient 16. For example, orientation marker 45 may also extend around the same portion of the circumference of lead body 48 or along the side of the paddle lead as electrodes 50. In this way, orientation marker 45 faces the same direction as electrodes, thus indicating the orientation of electrodes 50 to the clinician. When the clinician implants lead 14 in patient 16, orientation marker 45 may remain visible to the clinician.

Neurostimulator 12 delivers stimulation therapy via electrodes 50 of lead 14. In particular, electrodes 50 are electrically coupled to a therapy delivery module 40 of neurostimulator 12 via conductors within lead body 48. In one embodiment, an implantable signal generator or other stimulation circuitry within therapy delivery module 40 delivers electrical signals (e.g., pulses or substantially continuous-time signals, such as sinusoidal signals) to targets stimulation site 18 (FIG. 1A) via at least some of electrodes 50 under the control of a processor 42. The implantable signal generator may be coupled to power source 47. Power source 47 may take the form of a small, rechargeable or non-rechargeable battery, or an inductive power interface that transcutaneously receives inductively coupled energy. In the case of a rechargeable battery, power source 47 similarly may include an inductive power interface for transcutaneous transfer of recharge power.

The stimulation energy generated by therapy delivery module 40 may be formulated as neurostimulation energy, e.g., for treatment of any of a variety of neurological disorders, or disorders influenced by patient neurological response. The electrical signals may be delivered from therapy delivery module 40 to electrodes 50 via a switch matrix and conductors carried by lead 14 and electrically coupled to respective electrodes 50.

Processor 42 may include a microprocessor, a controller, a DSP, an ASIC, an FPGA, discrete logic circuitry, or the like. Processor 42 controls the implantable signal generator within therapy delivery module 40 to deliver neurostimulation therapy according to selected stimulation parameters. Specifically, processor 42 controls therapy delivery module 40 to deliver electrical signals with selected amplitudes, pulse widths (if applicable), and rates specified by the programs. In addition, processor 42 may also control therapy delivery module 40 to deliver the neurostimulation signals via selected subsets of electrodes 50 with selected polarities. For example, electrodes 50 may be combined in various bipolar or multi-polar combinations to deliver stimulation energy to selected sites, such as nerve sites adjacent the spinal column, pelvic floor nerve sites, or cranial nerve sites.

Processor 42 may also control therapy delivery module 40 to deliver each signal according to a different program, thereby interleaving programs to simultaneously treat different symptoms or provide a combined therapeutic effect. For example, in addition to treatment of one symptom such as sexual dysfunction, neurostimulator 12 may be configured to deliver neurostimulation therapy to treat other symptoms such as pain or incontinence.

Memory 44 of neurostimulator 12 may include any volatile or non-volatile media, such as a RAM, ROM, NVRAM, EEPROM, flash memory, and the like. In some embodiments, memory 44 of neurostimulator 12 may store multiple sets of stimulation parameters that are available to be selected by patient 16 via patient programmer 28 (FIG. 1) or a clinician via clinician programmer 26 (FIG. 1) for delivery of neurostimulation therapy. For example, memory 44 may store stimulation parameters transmitted by clinician programmer 26 (FIG. 1). Memory 44 also stores program instructions that, when executed by processor 42, cause neurostimulator 12 to deliver neurostimulation therapy. Accordingly, computer-readable media storing instructions may be provided to cause processor 42 to provide functionality as described herein.

In particular, processor 42 controls telemetry module 46 to exchange information with an external programmer, such as clinician programmer 26 and/or patient programmer 28 (FIG. 1), by wireless telemetry. In addition, in some embodiments, telemetry module 46 supports wireless communication with one or more wireless sensors that sense physiological signals and transmit the signals to neurostimulator 12.

Migration of lead 14 following implantation may be undesirable, and may have detrimental effects on the quality of therapy delivered to a patient 16. For example, with respect to the sacral nerve stimulation application shown in FIG. 1A, migration of lead 14 may cause displacement of electrodes carried by lead 14 to a target stimulation site 18. As a result, the electrodes may not be properly positioned to deliver the therapy to target stimulation site 18, resulting in reduced electrical coupling, and possibly undermining therapeutic efficacy of the neurostimulation therapy from system 10. Substantially fixing lead 14 to surrounding tissue may help prevent lead 14 from migrating from target stimulation site 18 following implantation, which may ultimately help avoid harmful effects that may result from a migrating neurostimulation lead 14.

To that end, lead 14 further includes inflatable balloon 54 mounted on lead body 48 distal to electrodes 50. As described above, inflatable balloon 54 may be mounted to lead body 48 to fixate lead 14 to tissue surrounding lead 14, such as tissue within sacrum 24 in the example of FIG. 1A or tissue at occipital region 29 in the example of FIG. 1B. As described in further detail below, inflatable balloon 54 may be expanded (i.e., inflated) by a fluid that is delivered to inflatable balloon 54 via one or more lumens extending generally from proximal end 48A of lead body 48 to inflatable balloon 54. The fluid delivery lumen may be disposed within lead body 48 or may be disposed outside of lead body 48.

While in the embodiment shown in FIG. 2, a single inflatable balloon 54 is located distal to electrodes 50, in other embodiments, a lead may include any suitable number of inflatable balloons in any suitable arrangement with respect to electrodes 50. For example, in one embodiment, inflatable balloon 54 may be mounted (or otherwise coupled) to lead body 48 proximate to electrodes 50 on lead body 48, between electrodes 50 and distal end 48B of lead body 48, between individual electrodes 50A-50D, and/or between electrodes 50 and proximal end 48A of lead body 48 in order to substantially fix electrodes 50 proximate to target stimulation site 18.

As discussed in further detail below, in accordance with one embodiment of the invention, inflatable balloon 54 is made of a degradable, elastic, and biocompatible material, such as, but not limited to, an elastic copolymer or a degradable thermoplastic polymer. For example, in some embodiments, balloon 54 comprises poly(esters) based on polyactide (PLA), polyglycolide (PGA), polucaprolactone (PCL) and/or blended with polysiloxanes, poly (ortho ester) copolymers, and poly (phoepazenese) elastomers.

The degradation rate and conditions of the material may be controlled. For example, the material may be designed to degrade in vivo over a predetermined time period (e.g., in response to an exposure to a certain temperature or humidity level for a predetermined duration of time). In another embodiment, the degradation of the material may be responsive to fibrous tissue ingrowth or encapsulation that contacts the material (e.g., fibrous tissue may encapsulate balloon 54 and apply a pressure that eventually causes a failure of the material forming balloon 54, thereby “popping” balloon 54). The degraded material as well as the inflation fluid may be absorbed by the patient's body.

In one embodiment, for sacral applications, inflatable balloon 54 may be approximately sized to be expandable to a diameter sufficient to fix lead 14 within tissue site posterior to sacral foramen 22. Alternatively, inflatable balloon 54 may facilitate fixation of lead 14 within other tissues target sites, including the epidural region proximate the spine. In those cases, inflatable balloon 54 may be sized to expand to any of a variety of diameters appropriate for engagement of tissue within the desired target therapy delivery site.

In comparison to some existing methods of fixing implanted medical leads, such as suturing lead 14 to surrounding tissue, inflatable balloon 54 may permit implantation of lead 14 in patient 16 via a minimally invasive surgery, which may allow for reduced pain and discomfort for patient 16 relative to surgery, as well as a quicker recovery time.

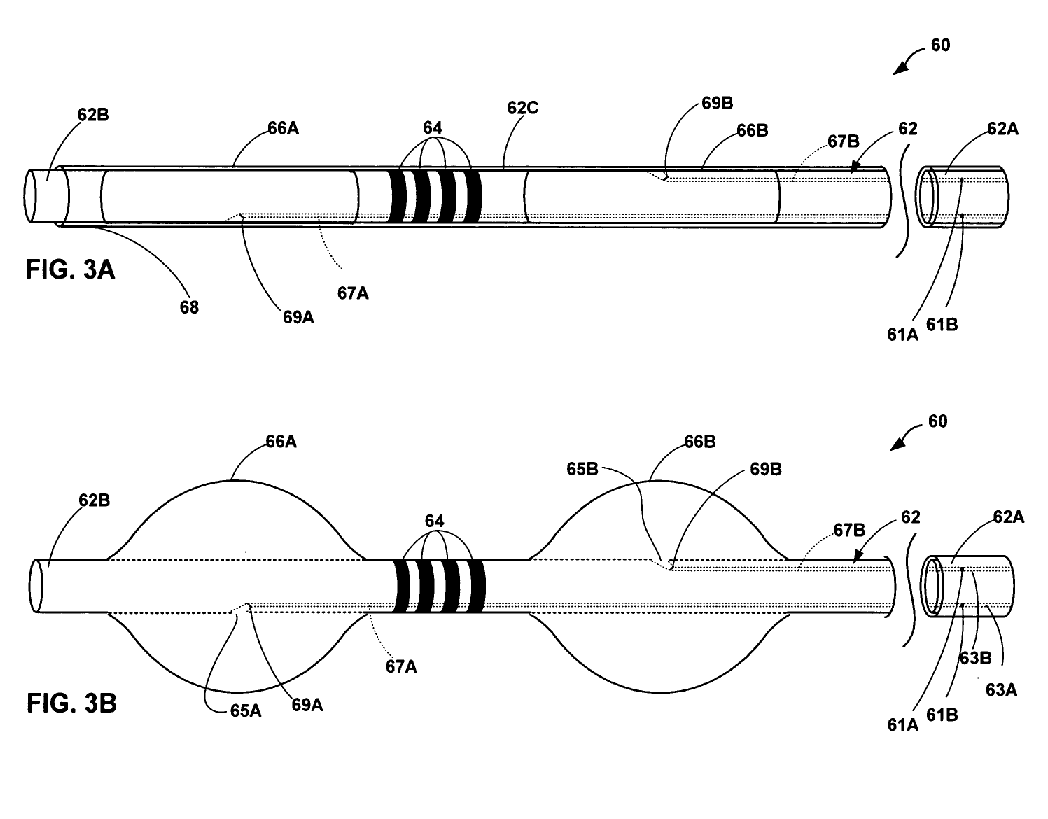

FIG. 3A is a perspective drawing illustrating an exemplary neurostimulation lead 60, which includes lead body 62 extending between proximal end 62A and distal end 62B, a plurality of stimulation electrodes 64, and balloons 66A and 66B (collectively “balloons 66”). Located within lead body 62 are inflation lumens 67A and 67B (collectively “inflation lumens 67”), which are shown in phantom lines. Inflation lumen 67A is in fluidic communication with balloon 66A, while inflation lumen 67B is in fluidic communication with balloon 66B. Disposed between each lumen 67A and 67B and the respective balloon 66A and 66B are fluid valves 69A and 69B, respectively.

Proximal end 62A of lead body 62 contains electrical contacts (not shown in FIGS. 3A and 3B) that are used to electrically connect electrodes 64 of lead 60 to a lead extension or a neurostimulator (e.g., neurostimulator 12 in FIG. 1A). Lead body 62 and electrodes 64 are similar to lead body 48 and electrodes 50 of FIG. 2. Balloons 66 are configured to expand radially outward from lead body 62 in order to engage with surrounding tissue to help prevent migration of lead 60 from the target stimulation site. While “radially outward” is referred to throughout the disclosure, it should be understood that the expansion of balloons 66 includes both axial and radial components because balloons 66 may extend from lead body 62 at an acute angle with respect to outer surface 62C of lead body 62.

In practice, balloons 66 facilitate fixation of neurostimulation lead 60 to surrounding tissue, e.g., within or posterior to sacral foramen 22 (FIG. 1A). Balloons 66 may be sized to be expandable to a diameter sufficient to fixate lead 60 within a target stimulation (or other therapy delivery) site. For example, balloons 66 may each be expandable to a diameter in a range of approximately 2 millimeters (mm) to 10 mm, and in one embodiment, approximately 4 mm to 6 mm, when disposed within a tissue site proximate the sacral foramen 22 in the presence of compressive forces generated by typical tissue. In another embodiment, balloons 66 may facilitate fixation of neurostimulation lead 60 to tissue surrounding neurostimulation lead 60 in other target stimulation sites. If lead 60 is implanted in the epidural region around the spine, for example, balloons 66 may each be expandable to a diameter in a range of approximately 6 mm to 15 mm, and in one embodiment, approximately 9 mm to 12 mm. In each scenario, a predetermined amount of fluid may be added to balloons 66 to expand balloons 66 to the desired dimension.

As described above, neurostimulation lead 60 carries a number of stimulation electrodes 64 to permit delivery of electrical stimulation to a target stimulation site such as a sacral nerve (FIG. 1A) or an occipital nerve (FIG. 1B). Accordingly, lead body 62 of neurostimulation lead 60 includes one or more conductors to electrically couple electrodes 64 to terminals within neurostimulator 12 (FIG. 1A). While four electrodes 64 are shown in the embodiment of lead 60 of FIG. 3A, in other embodiments, lead 60 may include any suitable number of electrodes.

Prior to implantation of lead 60 in patient 16, balloons 66 are each in a first, substantially deflated state as shown in FIG. 3A and have a first dimension. Balloons 66 are shown in a substantially deflated state in FIG. 3A. Balloons 66 may be introduced into patient 16 in the substantially deflated state during implantation to permit lead 60 to retain a relatively small overall profile. Lead 60 may be deployed via a needle or other minimally invasive delivery device. Introducing lead 60 via an introducer needle requires only minimally invasive techniques, which allows for a quicker recovery. Furthermore, by minimizing the overall profile of lead 60 during implantation in patient 16, the diameter of the introducer needle or other introducing device may be minimized, thereby minimizing the invasiveness of the implantation procedure.

In a deflated state, balloons 66 may be substantially flush with lead body 62. For example, balloons 66 may be disposed within recesses within lead body 62 or otherwise coupled to the outer surface 62C of lead body 62. In alternative embodiments, portions of balloons 66 may slightly protrude from lead body 62 in their deflated states. In both cases, restraint 68 may be used to protect, and if necessary restrain, balloons 66 in their deflated state. In some embodiments, the lead introducer may function as restraint 68.

The “deflated” and “inflated” states are relative to each other. For example, as used herein, “deflated” does not necessarily mean that balloons 66 are each 100% devoid of an inflating fluid, but that balloons 66 are each more devoid of fluid than in the “inflated” state. Or from the perspective of the inflated state, “inflated” does not necessarily mean that balloons 66 are each completely filled with fluid, but that balloons 66 each include more fluid than in the “deflated state.”

In one embodiment, at least a portion of neurostimulation lead 60, such as lead body 62, may include radio-opaque material that is detectable by imaging techniques, such as fluoroscopic imaging or x-ray imaging. This feature may be helpful for maneuvering neurostimulation lead 60 relative to a target site within the body. For example, the distal end 62B of neurostimulation lead 60 may include radio-opaque material that is visible via fluoroscopic imaging. Radio-opaque markers, as well as other types of markers, such as other types of radiographic and/or visible markers, may also be employed to assist a clinician during the introduction and withdrawal of neurostimulation lead 60 from a patient.

Upon implantation in patient 16, a fluid, such as, but not limited to, water or a saline solution, may introduced into balloons 66 via inflation lumens 67A and 67B such that balloons 66 each expand to a second, expanded state and extend past outer surface 62C of lead body 62 to engage with surrounding tissue. FIG. 3B is a perspective drawing illustrating an exemplary neurostimulation lead 60 with balloons 66 in an expanded state, in which balloons 66 extend from lead body 62 to enable balloons 66 to engage with surrounding tissue to substantially fix neurostimulation lead 60 proximate to target stimulation site 18.

In the expanded, inflated state, balloons 66 each have a second dimension, which is greater than the first dimension in the unexpanded, deflated state, thereby enlarging the profile of at least a portion of lead 60. Just as with balloon 54 of lead 14 of FIG. 2, balloons 66 may be expandable to any suitable diameter, which may depend on the particular stimulation application of lead 60. By engaging with surrounding tissue, balloons 66 help substantially fix a position of lead 60 to (e.g., at or near) target stimulation site 18, thereby reducing migration of lead 60. While balloons 66 do not necessarily restrict all motion of lead 60 when balloons 66 are in the inflated state, balloons 66 generally reduce the motion of lead 60 so that lead 60 remains proximate to target stimulation site 18.

In the expanded, inflated state, balloons 66 may define protrusions of any suitable shape and size that are capable of engaging with surrounding tissue when implanted in patient 16. In the embodiment of lead 60 shown in FIGS. 3A and 3B, balloons 66 each define a round structure. In other embodiments, balloons 66 may define tines or flange-like structures when expanded.

After balloons 66 have been inflated, inflation lumens 67 may be sealed at proximal end 62A of lead body 62 to prevent fluid from leaking out of inflation lumens 67. For example, septa 61A and 61B (collectively “septa 61”), e.g., silicone seal-sealing ports, may used to seal inflation lumens 67A and 67B at their proximal ends 63A and 63B (collectively “proximal ends 63”), respectively. Septa 61 may be penetrated by fine needles and sealed automatically following removal of the needles, e.g., silicone components may self-seal upon removal of the needles. Additionally or alternatively, inflation lumens 67A and 67B may include fluid valves 69A and 69B (collectively “valves 69”) at the entrance to balloons 66A and 66B, respectively. Valves 69 may each be one-way valves that are configured to open when fluid is introduced into inflation lumens 67 and allow the fluid to flow into balloons 66. That is, the force of fluid within inflation lumens 67A and 67B may force the respective valve 69A and 69B open, thereby allowing the fluid to flow into balloons 66A and 66B, respectively. In one embodiment, valves 69A and 69B also seal interfaces 65A and 65B, respectively, between the respective balloons 66A and 66B and inflation lumens 67A and 67B once balloons 66 are filled with fluid in order to minimize fluid leakage from balloons 66 into lumens 67. For example, valves 69 may each be biased (e.g., by a spring or material properties) toward the respective inflation lumen 67, such that valves 69 automatically close and seal the respective interface when fluid is no longer flowing through inflation lumens 67 and into the respective balloons 66. Examples of suitable types of valves 69 include butterfly valves or flap-shaped valves with stops for controlling the direction the flaps may open.

In other embodiments, a series of seals may be provided in order to help retain fluid within expanded balloons 66 by both sealing inflation lumens 67 at proximal end 62A of lead body 62 and using valves 69. A series of seals may be used as a precaution in case one of the seals malfunctions.

Balloons 66 may be made out of a material that deteriorates over time, during which time, fibrous ingrowth may secure the lead to prevent migration. For example, balloons 66 may be made out of a degradable thermoplastic polymer or any other degradable, elastic, and biocompatible material. In one embodiment, balloons 66 are made out of an elastic copolymer designed to degrade in vivo over a predetermined amount of time. The composition of the elastic copolymer, i.e., the percent composition of each of the polymer components, may be adjusted to influence elastic and mechanical properties as well as the rate of degradation. Additionally, the degradation of the balloon material may be responsive to fibrous tissue ingrowth that contacts the material. Specifically, as fibrous tissue grows into the balloon and the lead, the material of the balloon 66 may be under increased tension, triggering the material to degrade at an increased rate. In instances of significant fibrous tissue ingrowth, balloons 66 may rupture under the force exerted on balloons 66 by the fibrous ingrowth. In a preferred embodiment, balloons 66 made of a degradable material are filled with a biocompatible fluid, e.g., water or saline solution, which is released into patient 16 as the material forming balloons 66 degrades or ruptures. The degraded material may be absorbed by the patient's body.

Therapy may require that the neurostimulation lead be activated for only a short period of time, e.g., for trial stimulation, sometimes referred to as screening. On the other hand, therapy may require that the neurostimulation lead be implanted chronically for a number of years. In either case, it may become necessary to remove (or “explant”) neurostimulation lead 60 from patient 16.

If balloons 66 are made of a material designed to deteriorate over a predetermined amount of time, balloons 66 may degrade and deflate prior to explantation from patient 16. However, in other cases in which balloons 66 are intact and inflated when lead 60 is to be removed from a patient, such as if the material forming balloons 66 does not deteriorate as designed or if lead 60 is explanted prior to the amount of time required for the deterioration of balloons 66, it may be desirable to deflate balloons 66 prior to explant. In one embodiment, balloons 66 are deflated by inserting a stylet into balloons 66 via inflation lumens 67 and rupturing balloons 66, which allows the fluid to be released into patient 16. In an alternative embodiment, the fluid is removed from balloons 66 via inflation lumens 67 by applying suction to generate a flow of fluid from balloons 66, through inflation lumens 67, and out proximal ends 63 of inflation lumens 67 near proximal end 62A of lead body 62. In embodiments in which inflations lumens 67 contain valves 69, the valves may be opened by introducing stylets into proximal ends 63 of inflation lumens 67 and through valves 69 prior to applying suction. Similarly, in embodiments in which inflation lumens 67 each contain a septum, the septa may be penetrated using stylets, needles, or any other appropriate tool to aid in deflation.

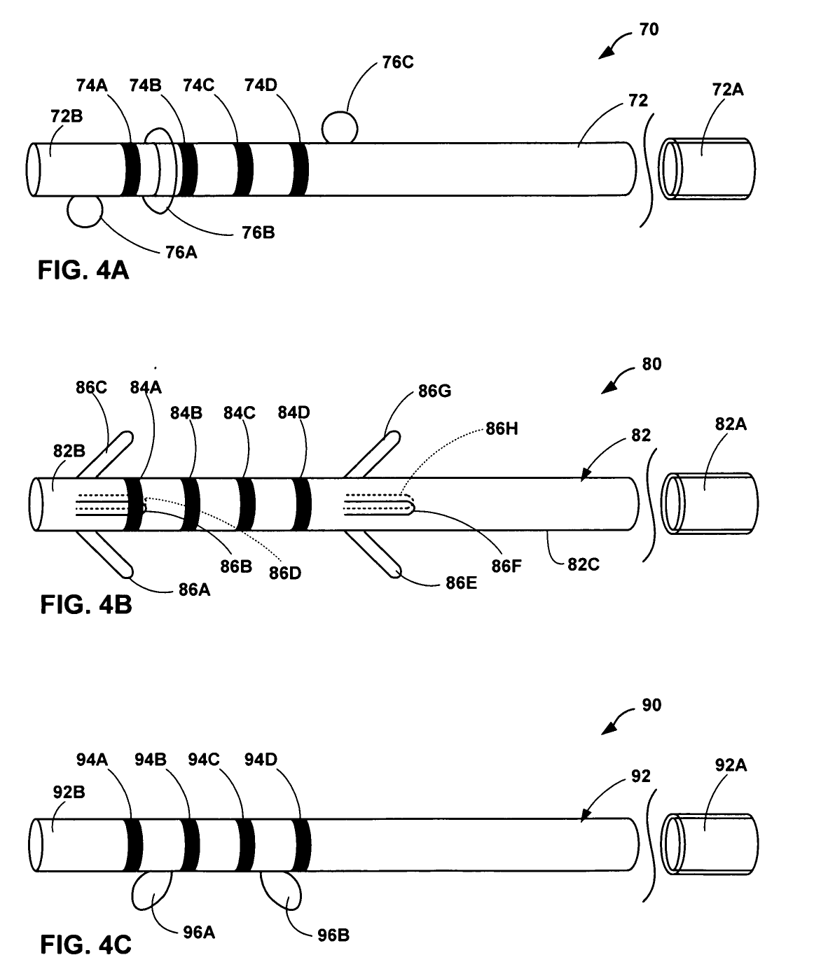

FIGS. 4A-4C are perspective views of leads including alternate configurations and arrangements of inflatable balloon fixation elements for substantially fixing positions of their respective leads in accordance with the invention. The leads illustrated in FIGS. 4A-4C are shown in their inflated state but are capable of being deflated and inflated using one or more inflation lumen (not shown in FIGS. 4A-4C), as previously described. Additionally, the proximal end of each lead body contains contacts (not shown in FIGS. 4A-4C) that are used to electrically connect each lead to a lead extension or a neurostimulator (e.g., neurostimulator 12 in FIG. 1A).

FIG. 4A illustrates an embodiment of lead 70, which includes lead body 72 extending between proximal end 72A and distal end 72B, and electrodes 74A-74D disposed proximate to distal end 72B of lead body 72. Lead 70 includes balloons 76A-76C (collectively “balloons 76”), which fix lead body 72 at both the proximal and distal side of electrodes 74A-74D and between two electrodes 74A and 74B. In particular, balloon 76A is located between distal end 72B of lead body 72 and electrodes 74A-74D (i.e., on the “distal side” of electrodes 74A-74D), balloon 76B is located between electrodes 74A and 74B, and balloon 76C is located between the proximal end 72A of lead body 72 and electrodes 74A-74D (i.e., on the “proximal side” of electrodes 74A-74D). An inflation lumen (e.g., inflation lumen 67A in FIGS. 3A-3B) may be fluidically connected to each of balloons 76 in order to provide a channel to deliver a fluid to balloons 76 to inflate balloons 76. Each balloon 76 may have its own inflation lumen or two or more balloons 76 may share an inflation lumen.

Balloons 76A-C each have a different configuration than balloons 66 of lead 60 (FIGS. 3A-3B), and are examples of other suitable configurations (e.g., shapes, sizes, etc.) of balloon fixation elements that may be used to fix a lead in accordance with the invention. Balloon 76B in FIG. 4A extends around the entire outer perimeter of lead body 72A. In other embodiments, balloon 76B may be distributed around a portion of the periphery of lead body 72 rather than extending substantially around the entire periphery. For example, in contrast to balloon 76B, balloons 76A and 76C extend from a portion of the periphery of lead body 72 rather than extending substantially around the periphery.

As shown in FIG. 4A, balloons 76A and 76C extend from only one side of the lead body, rather than being distributed about the periphery of lead body 72. FIG. 4A further illustrates an embodiment of lead 70 in which balloons 76A and 76C located at different axial positions along lead body 72 extend from different sides of lead body 72. More specifically, FIG. 4A illustrates first balloon element 76A located at a first axial position extending in a first direction, and second balloon element 76C located at a second axial position extending in a second direction that differs from the first direction. In FIG. 4A, balloons 76A and 76C extend in approximately opposite directions. However, in other embodiments, balloons 76A and 76C may each extend in directions that are not approximately opposite each other.

While fixing a lead at either the proximal side or distal side of the electrodes may be useful in some applications, in other applications, it may be desirable to fix the lead at both the proximal and distal sides of the electrodes, as depicted in FIG. 4A. Balloons 76A and 76C located distally and proximally to the electrodes 74A-74D, respectively, may provide a more secure attachment than simply fixating lead 70 at one portion of lead body 72. For example, by fixing lead 70 on both the proximal and distal sides of electrodes 74A-74D, the portion of lead body 72 containing electrodes 74A-74D may remain more stationary. This may be useful, for example, in an application in which the lead (e.g., lead 70 of FIG. 4A) is a part of a therapy system delivering electrical stimulation to a pudendal nerve of a patient. Furthermore, fixating lead 70 between two electrodes 74A and 74B may more locally fix one or more of the electrodes to the surrounding tissue.

FIG. 4B illustrates another embodiment of lead 80, which includes lead body 82 extending between proximal end 82A and distal end 82B and electrodes 84A-84D disposed proximate to distal end 82B of lead body 82. Inflatable balloons 86A-86H are coupled to lead body 82 to substantially fix a position of lead 82 proximate to a target stimulation site. In the inflated state (as shown in FIG. 4B), each of balloons 86A-H defines a tine-like structure that protrudes from outer surface 82C of lead body 82. An inflation lumen (e.g., inflation lumen 67A in FIGS. 3A-3B) may be fluidically connected to each of balloons 86 in order to provide a channel to deliver a fluid to balloons 86 to inflate balloons 86. Each balloon 86 may have its own inflation lumen or two or more balloons 86 may share an inflation lumen.

As shown in FIG. 4B, balloons 86A-86D are located at a first axial position with respect to lead body 82, and balloon 86E-86H are located at a second axial position with respect to lead body 82. Balloons 86D and 86H are located on the circumferential portion of lead 80 not visible in FIG. 4B. The approximate locations of balloons 86D and 86H are outlined with phantom lines. Additionally, balloons 86A-86D may be, but need not be, evenly distributed around the periphery of lead body 82. Balloons 86A-86D are located on a portion of lead body 82 proximal to electrodes 84A-84D, and balloons 86E-86H are located on a portion of lead body 82 distal to electrodes 84A-84D. More specifically, balloons 86A-86D are disposed between the most distally located electrode 84A and distal end 82B of lead body 82, and balloons 86E-86H are disposed between the most proximally located electrode 84D and proximal end 82A of lead body 82. Alternatively, one or more balloon elements may be disposed in between individual electrodes 84A-84D, e.g. between electrodes 84A and 84B.

Balloons 86A-86D and 86E-86H (collectively “balloons 86”), shown in FIG. 4B, are angled in their expanded states such that they have both a radial and axial component. In particular, balloons 86 each extend from lead body 82 at an acute angle with respect to outer surface 82C of lead body 82. As shown in FIG. 4B, balloons 86 are angled toward proximal end 82A of lead body 82. Angling balloons 86 toward proximal end 82A of lead body 82 may aid in limiting migration of lead 80 toward the direction in which the balloons are angled, i.e., toward proximal end 82A. In other embodiments, lead 80 may include balloons that also extend toward distal end 82B when inflated, or alternatively, lead 80 may only include balloons that extend toward distal end 82B.

As an additional alternative, a lead may only include balloon elements between electrodes to ensure fixation of the one or more electrodes proximate to target stimulation site 18, as shown in FIG. 4C. FIG. 4C is a perspective view of lead 90, which includes lead body 92, electrodes 94A-94D, balloon 96A located between electrodes 94A and 94B, and balloon 96B located between electrodes 94C and 94D. This configuration may locally fixate electrodes 94B and 94C as well as generally fixate lead 90. Locally fixating electrodes 94B and 94C may useful in applications where a clinician aims to implant lead 90 such that the mid-length of the electrode region of lead body 92, i.e., the location between electrodes 94B and 94C, is centered at target stimulation site 18. Alternatively, balloons 96A and 96B may be positioned to locally secure various electrodes (e.g., between electrodes 94B and 94C). An inflation lumen (e.g., inflation lumen 67A in FIGS. 3A-3B) may be fluidically connected to each of balloons 96 in order to provide a channel to deliver a fluid to balloons 96 to inflate balloons 96. Each balloon 96 may have its own inflation lumen or two or more balloons 96 may share an inflation lumen.

In FIG. 4C, balloon 96A on lead 90 is shown angled toward distal end 92B of lead body 92 and away from electrode 94B, and balloon 96B is shown angled toward proximal end 92A of lead body 92 and away from electrode 94C. If a clinician aims to implant lead 90 such that the mid-length of the electrode region of lead body 92, i.e., the location between electrodes 94B and 94C, is centered at target stimulation site 18, it may be desirable to angle balloons 96A and 96B away from the mid-length of the electrode region. This configuration may allow electrodes 94B and 94C to have more direct contact with the target stimulation site.

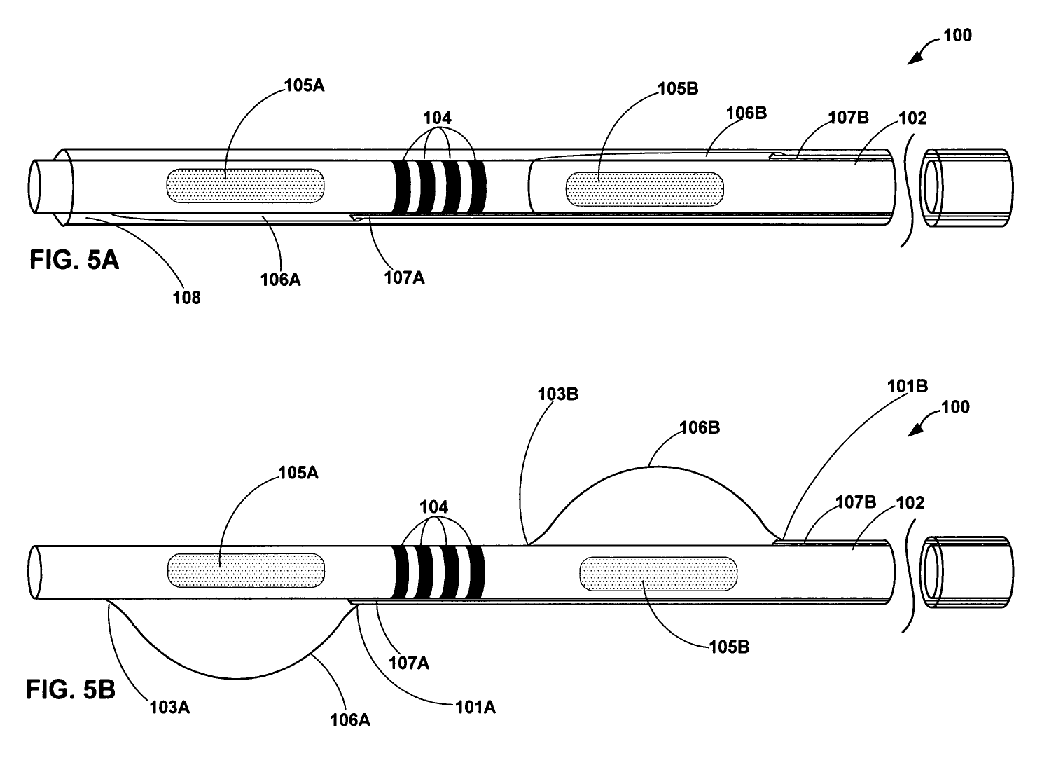

FIGS. 5A and 5B are perspective views of an alternative inflation lumen configuration that may be used to inflate balloon fixation elements in accordance with one embodiment of the invention. Lead 100 includes lead body 102, electrodes 104, and balloons 106A and 106B (collectively “balloons 106”). FIG. 5A shows balloons 106 in a deflated state within restraint 108, and FIG. 5B shows balloons 106 in an inflated state. Inflation lumens 107A and 107B (collectively “inflation lumens 107”) define channels for delivering fluid to balloons 106A and 106B, respectively. In contrast to inflation lumens 67 of FIGS. 3A and 3B, inflation lumens 107 are disposed outside of lead body 102. Inflation lumens 107 may be attached to lead body 102 along the entire length of lead body 102 or at intermittent points along lead body 102.

In the embodiment illustrated in FIGS. 5A and 5B, a portion of proximal ends 101A and 101B of balloons 106A and 106B are attached to inflation lumens 107A and 107B, respectively. Distal ends 103A and 103B of balloons 106A and 106B, respectively, are attached to lead body 102. Attaching proximal ends 101A and 101B of balloons 106A and 106B to inflation lumens 107A and 107B, respectively, serves to seal balloons 106 against inflation lumens 107 such that balloons 106 will be capable of holding water and expanding.

Additionally, lead 100 of FIGS. 5A and 5B contains porous portions 105A and 105B (collectively “porous portions 105”). Porous portions 105 may promote fibrous ingrowth into lead 100, which may aid in fixing lead 100. Porous portions 105 may be composed of expandable poly-tetra-fluoro-ethylene (ePTFE) or any other suitable biocompatible material. Any portion of lead body 102 or any portion of any elongated member in accordance to the invention may be porous.

In general, one or more balloons formed of a biodegradable material may be used in fixating a lead in accordance with the invention. Additionally, other forms of fixation elements may be used in addition to balloons. The additional fixation elements may be any suitable actively or passively deployed fixation element that helps prevent migration of lead 100 when lead 100 is implanted in patient 16, such as, but not limited to, one or more tines, barbs, hooks, wire-like elements, adhesives (e.g., surgical adhesives), balloon-like fixation elements, pinning fixation elements, collapsible or expandable fixation structures, and so forth. The fixation elements may be composed of any suitable biocompatible material, including, but not limited to, polymers, titanium, stainless steel, Nitinol, other shape memory materials, hydrogel or combinations thereof. For example, tines may be added to a lead body of any of the illustrated embodiments to provide a more secure fixation. Examples of suitable tines include, but are not limited to, the tines described in commonly-assigned U.S. Pat. No. 6,999,819, entitled, “IMPLANTABLE MEDICAL ELECTRICAL STIMULATION LEAD FIXATION METHOD AND APPARATUS,” which issued on Feb. 14, 2006 and is hereby incorporated by reference in its entirety. If additional fixation elements are used in addition to one or more balloons, all of the fixation elements may be restrained during implantation of the lead and expanded upon implantation. Also, all of the fixation mechanisms may be configured to permit explant.

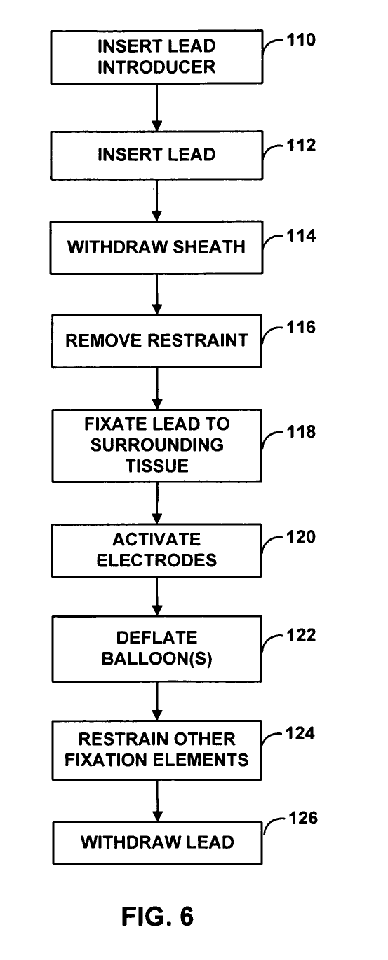

FIG. 6 is a flow diagram illustrating a process for percutaneously implanting a lead including a balloon fixation mechanism in accordance with one embodiment of the invention. While the process shown in FIG. 6 is described with respect to lead 60 of FIGS. 3A and 3B, in other embodiments, the lead may be, for example, any one of leads 14, 70, 80, 90 or 100 of FIGS. 2, 4A-4C, and 5A, respectively. In addition, the process shown in FIG. 6 may be used to implant any suitable lead including a fixation mechanism including one or more expandable balloon elements formed at least in part of a biodegradable material in accordance with the invention. Furthermore, while the process is described with reference to percutaneously implanting lead 60 proximate to target stimulation site 18 of FIG. 1A, in other embodiments, lead 60 may be implanted proximate to any suitable target stimulation site or target therapy delivery site.

Initially, an introducer needle assembly is inserted into patient 16 (110) and guided through sacral foramen 22 of sacrum 24 to target neurostimulation site 18. The needle assembly may include a needle and an introducer stylet fitted into a lumen defined by the needle. In one embodiment, the lumen has a diameter between 14 and 20 gauge to allow the needle to receive the introducer stylet. The introducer stylet may fill the lumen of the needle, preventing tissue coring. In some instances, the needle may include a straight needle for sacral implantation or a modified Tuohy needle for epidural applications, which has an opening that is angled approximately 45 degrees so that an instrument passing through the needle exits at an angle.

The neurostimulation lead introducer may be inserted (110) by a variety of techniques not limited to the technique described above. Lead 60 is inserted (112) and advanced through the lead introducer. Lead 60 is typically advanced through the introducer until electrodes 50 reach tissue proximate to the target stimulation site. Meanwhile, balloons 66 are in a deflated state and restraint mechanism 68 may protect the balloon material from damage. Restraint mechanism 68 may also serve to restrain other expandable fixation elements that may optionally be included on the lead 60. In other embodiments, the restraint mechanism may be the lead introducer, a sheath other than the lead introducer, or the like. Once the neurostimulation lead reaches target stimulation site 18, the lead introducer is withdrawn (114). In one embodiment, the restraint mechanism includes the lead introducer. In this case, the act of withdrawing the lead introducer exposes balloons 66 and removes the restraint on any additional fixation elements. Alternatively, the restraint mechanism 68 may need to be removed after the lead introducer (116).

After the neurostimulation lead 60 has been properly placed proximate to target stimulation site 18, balloons 66 are inflated to allow balloons 66 to extend from lead body 62 and engage with surrounding tissue to fixate lead 60 proximate to target stimulation site 18 (118). Balloons 66 may be inflated by introducing (e.g., injecting) a fluid, such as water, saline solution, or another biocompatible fluid, through inflation lumens 67. Fixating lead 60 to surrounding tissue may prevent detrimental effects that may result from a neurostimulation lead 60 migrating.

Electrodes 64 on lead 60 may be activated (120) to provide therapy to the patient, e.g., by coupling a proximal end 62A of neurostimulation lead body 62 to a neurostimulator (e.g., neurostimulator 12 of FIGS. 1 and 2). In one embodiment, a lead extension may be provided to couple the neurostimulation lead to the neurostimulator.

Therapy may require that electrodes 64 of neurostimulation lead 60 be activated for only a short period of time, e.g., for trial stimulation, sometimes referred to as screening. On the other hand, therapy may require that lead 60 be implanted chronically for a number of years. In either case, it may become necessary to remove neurostimulation lead 60 from patient 16. In order to aid explantation of neurostimulation lead 60, balloons 66 may be deflated (122), and if other fixation elements were included on the lead body, they may be restrained as they were when the lead was inserted or otherwise disengaged from surrounding tissue (124).

If balloons 66 are made of a material designed to deteriorate over a predetermined amount of time, balloons 66 may degrade and deflate prior to explantation from patient 16, so it may not be necessary to deflate balloons 66 (122). However, in other cases in which balloons 66 are intact and inflated when lead 60 is to be removed from a patient, it may be desirable to deflate balloons 66 prior to explant. In one embodiment, balloons 66 are deflated by inserting a stylet into balloons 66 via inflation lumens 67 and rupturing balloons 66, which allows the fluid to be released into the patient. In an alternative embodiment, the fluid is removed from balloons 66 via inflation lumens 67 using suction. In embodiments in which inflations lumens 67 contain valves 69, the valves may be opened by inserting stylets into inflation lumens 67 to open valves 69 prior to applying suction. Once all of the fixation elements are deflated or restrained, neurostimulation lead 60 may be withdrawn from patient 16 (126).

A lead including degradable balloon fixation elements in accordance with the invention may be useful for various electrical stimulation systems. For example, the lead may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. In addition, the fixation element arrangement described herein may also be useful for fixing a catheter, such as a drug deliver catheter, proximate to a target drug delivery site.

Many embodiments of the invention have been described. Various modifications may be made without departing from the scope of the claims. For example, the present invention further includes within its scope methods of making and using systems and leads for neurostimulation, as described herein, as well as methods of making and using elongated members for therapy systems. Also, the elongated members described herein may have a variety of therapy applications, such as fluid delivery to a target therapy delivery site or other electrical stimulation applications (e.g., sensing or delivery of cardiac electrical stimulation, including paces, pulses, and shocks). These and other embodiments are within the scope of the following claims.

1. An implantable elongated member comprising:
an elongated body configured to be coupled to a medical device to deliver a therapy from the medical device to a target therapy delivery site in a patient;
an inflation lumen; and
a balloon fixation element coupled to the elongated body and composed at least in part of a biocompatible degradable material, the balloon fixation element being configured to receive a fluid via the inflation lumen to expand from a first state to a second state.

2. The apparatus of claim 1, wherein the inflation lumen is disposed within the elongated body.

3. The apparatus of claim 1, further comprising a fluid valve between the balloon fixation element and the inflation lumen.

4. The apparatus of claim 1, wherein the elongated member comprises a lead comprising a lead body extending between a proximal end and a distal end, and one or more electrodes proximate to the distal end of the lead body.

5. The apparatus of claim 1, wherein the medical device comprises at least one of a sensor to sense a parameter of a patient, an electrical stimulator or a fluid delivery device.

6. The apparatus of claim 1, wherein the elongated body comprises a catheter configured to deliver a fluid from the medical device to the target therapy delivery site.

7. The apparatus of claim 1, wherein the balloon fixation element is sized to be expandable to a diameter in a range of approximately 2 millimeters to 15 millimeters.

8. The apparatus of claim 1, further comprising a radio-opaque material that is detectable by fluoroscopic imaging located on at least a portion of the elongated member.

9. The apparatus of claim 1, wherein the fluid is a biocompatible fluid, and wherein the biocompatible fluid is released into the patient as the material of the balloon fixation element degrades.

10. The apparatus of claim 1, wherein the biocompatible degradable material of the balloon fixation elements is at least one of an elastic copolymer or a thermoplastic polymer.

11. The apparatus of claim 1, wherein the balloon fixation element is configured to deflate from the second state to the first state by removing fluid via the inflation lumen.

12. The apparatus of claim 1, wherein fibrous tissue growth around the elongated body and the balloon fixation element aids in degrading the biocompatible degradable material.

13. The apparatus of claim 1, further comprising a porous portion on the elongated body, wherein the porous portion promotes fibrous tissue ingrowth.

14. A system comprising:
a medical device; and
an elongated member comprising:
an implantable elongated body configured to be coupled to a medical device to deliver a therapy from the medical device to a target therapy delivery site in a patient;
an inflation lumen; and
a balloon fixation element coupled to the elongated body and composed at least in part of a biocompatible degradable material, the balloon fixation element being configured to receive a fluid via the inflation lumen to expand from a first state to a second state.

15. The system of claim 14, wherein the elongated member further comprises a fluid valve between the balloon fixation element and the inflation lumen.

16. The system of claim 14, wherein the elongated member comprises at least one of a lead comprising an electrode and the medical device comprises an electrical stimulator coupled to the proximal end of the elongated member and configured to deliver electrical stimulation to the target therapy delivery site via the electrode of the lead.

17. The system of claim 14, wherein the elongated element comprises a catheter, and wherein the medical device comprises a fluid delivery pump coupled to deliver a fluid to the target tissue via the catheter.

18. The system of claim 14, further comprising a restraint mechanism to protect the biocompatible degradable material of the balloon fixation element, wherein the restraint mechanism is removed prior to expanding the balloon fixation element from the first state to the second state.

19. The system of claim 14, wherein the biocompatible degradable material of the balloon fixation elements is at least one of an elastic copolymer or a thermoplastic polymer.

20. The system of claim 14, further comprising a porous portion on the elongated member, wherein the porous portion promotes fibrous tissue ingrowth.

21. A method comprising:
inserting an elongated member into the patient, wherein the elongated member includes a balloon fixation element mounted to the elongated member and composed at least in part of a biocompatible degradable material;
advancing the elongated member to a target therapy delivery site to deploy the balloon fixation element into tissue of the patient proximate to the target therapy delivery site; and
delivering a fluid to the balloon fixation element via an inflation lumen to inflate the balloon fixation element from a first state to a second state, wherein in the second state, the balloon fixation element engages with tissue at the target therapy delivery site.

22. The method of claim 21, wherein the inflation lumen is disposed within the elongated member.

23. The method of claim 21, wherein inserting the elongated member into the patient comprises inserting an introducer into a patient.

24. The method of claim 21, wherein inserting the introducer into the patient comprises introducing the introducer proximate to a peripheral nerve of the patient.

25. The method of claim 24, wherein inserting the introducer proximate to the peripheral nerve comprises positioning the introducer substantially transversely across an occipital nerve.

26. The method of claim 21, wherein the elongated member comprises at least one of a lead comprising an electrode or a catheter.

27. The method of claim 21, further comprising coupling the elongated member to a medical device, the medical device delivering a therapy to the target therapy delivery site via the elongated member, wherein the medical device is at least one of an electrical stimulator, a sensor or a fluid delivery device.

28. The method of claim 21, further comprising removing the fluid from the balloon fixation elements via the inflation lumens to deflate the balloon fixation element.
